(19) United States
(12) Patent Application Publication
(10) Pub. No.: US 2013/0245118 A1
(43) Pub. Date: Sep. 19, 2013

(54) STABLE FATTY ACID-CONTAINING FORMULATIONS

(71) Applicant: Eric H. Kuhrts, Novato, CA (US)

(72) Inventor: Eric H. Kuhrts, Novato, CA (US)

(21) Appl. No.: 13/808,079

(22) PCT Filed: Jul. 5, 2011
(86) PCT No.: PCT/US11/42970

§ 371 Date: May 21, 2013

Related U.S. Application Data
(60) Provisional application No. 61/361,069, filed on Jul. 2, 2010.

(51) Int. Cl. A23L 1/30; A61K 47/10; A61K 47/14

(52) U.S. Cl. CPC A23L 1/3008; A61K 47/14; A61K 47/10
USPC 514/560; 514/558; 426/648; 426/590; 426/2

(57) ABSTRACT

Methods and formulations for increasing the water solubility, stability, shelf life, and/or bioavailability of dietary fatty acids are disclosed. The formulation can comprise a dietary fatty acid, a non-ionic surfactant, a flavonoid or polyphenol, and optionally, water.

### BACKGROUND

Dietary or nutritional fatty acids are a family of unsaturated fatty acids that include the omega-3 fatty acids such as eicosapentaenoic acid (EPA) and docosahexaenoic acid (DHA), as well as omega-6 and omega-9 fatty acids. One of the primary sources for the omega-3 fatty acids is fish oil; however, omega-3 fatty acids can also be obtained from botanical sources and algae. With respect to omega-3 fatty acids, many cardiovascular and other health benefits are known, in addition to their significance in nutrition. In fact, consumption of nutritional or dietary fatty acids have been identified with many health benefits, having the potential to impact numerous diseases such as cardiovascular, neurological, immune function, and arthritis. Due to the increased awareness of the health benefits of the omega-3 class of fatty acids, dietary food supplements of fish oil and flax oil have become popular. With the availability of deodorized fish oils, it is now possible to make beverages containing omega-3 fatty acids, or fish oil, but the stability of the oils remains a problem. As such, it would be advantageous to provide a more stable, water-soluble formulation of these fatty acids for use in beverages. Such a product would have better shelf-life characteristics and more desirable sensory qualities for consumers.

### SUMMARY

The present disclosure relates to unique pharmaceutical compositions or water-soluble formulations of a dietary fatty acid, a non-ionic surfactant, a flavonoid or polyphenol, and water. In an alternative embodiment, the water-soluble formulation can be in the form of a stable, water soluble pharmaceutical gel comprising a dietary fatty acid, a non-ionic surfactant, and a flavonoid or polyphenol.

In another embodiment, a method of stabilizing dietary fatty acids in water can comprise warming a non-ionic surfactant; adding a flavonoid or a polyphenol to the non-ionic surfactant and mixing until dissolved; combining a dietary fatty acid with the non-ionic surfactant and flavonoid or polyphenol to form a surfactant-dietary fatty acid-flavonoid or polyphenol mixture; and combining the surfactant-dietary fatty acid-flavonoid or polyphenol mixture with water to form stabilized, clear, water-soluble, self-assembled fatty acid solution.

In another embodiment, a method of making a stable, water-soluble pharmaceutical gel composition of dietary fatty acids can comprise heating a water-soluble non-ionic surfactant in a container to a temperature of about 90° F. to about 200° F. while mixing the non-ionic surfactant until a clear non-ionic surfactant is formed; and adding a flavonoid or polyphenol to the clear non-ionic surfactant and mixing until a clear non-ionic surfactant-flavonoid or polyphenol combination is formed. An additional step can include adding a dietary fatty acid to the clear non-ionic surfactant-flavonoid or polyphenol combination and stirring until thoroughly mixed so as to constitute from 0.1 wt % to 25 wt % dietary fatty acid, from 70 wt % to 99.9 wt % surfactant, and from 0.01 wt % to 5 wt % flavonoid or polyphenol, wherein the dietary fatty acid and flavonoid or polyphenol is sufficiently dispersed or dissolved in the surfactant so that a gel composition is formed containing no visible micelles or particles of dietary fatty acid.

In another embodiment, a method of enhancing the stability of a dietary fatty acid in a beverage can comprise combining a dietary fatty acid, a non-ionic surfactant with a flavonoid, and water, to form a surfactant-dietary fatty acid-flavonoid-water mixture.

In yet another embodiment, a method of delivery a dietary fatty acid to a subject can comprise administering a water-soluble formulation of a dietary fatty acid, a non-ionic surfactant, a flavonoid or polyphenol, and water in the form of a beverage to a subject.

### DETAILED DESCRIPTION

### I. Definitions

Before the present invention is disclosed and described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

In describing and claiming the present invention, the following terminology will be used in accordance with the definitions set forth below.

It is noted that, as used herein, the singular forms of “a,” “an,” and “the” include plural referents unless the context clearly dictates otherwise. Thus, for example, reference to “a dietary fatty acid” includes one or more of such dietary fatty acids.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The abbreviations used herein have their conventional meaning within the chemical and biological arts.

“Dietary fatty acid” as used herein, includes one or more nutritional fatty acid, such as omega-3 fatty acids derived from natural sources such as fish, algae, or botanical sources such as Chia, Sage, Salvia hispanica, or Flax sources derived from linseed, or produced synthetically. The following is a list of omega-3 fatty acids (Table 1) followed by a list of botanical extracts of omega-3 fatty acids (Table 2). These lists are exemplary only, and are not considered to be limiting.

TABLE 1
List of several common n-3 fatty acids found in nature

| | Lipid | |
|---|---|---|
| Common Name | Name | Chemical Name |
| — | 16:3 (n-3) | all-cis-7,10,13- |
| | | hexadecatrienoic acid |
| Alpha-Linolenic acid (ALA) | 18:3 (n-3) | all-cis-9,12,15- |
| | | octadecatrienoic acid |
| Stearidonic acid (STD) | 18:4 (n-3) | all-cis-6,9,12,15- |
| | | octadecatetraenoic acid |
| Eisosatrienoic acid (ETE) | 20:3 (n-3) | all-cis-11,14,17- |
| | | eicosatrienoic acid |
| Eicosatetraenoic acid (ETA) | 20:4 (n-3) | all-cis-8,11,14,17- |
| | | eicosatrienoic acid |
| Eicosapentaenoic acid (EPA) | 20:5 (n-3) | all-cis-5,8,11,14,17- |
| | | eicosapentaenoic acid |
| Docosapentaenoic acid | 22:5 (n-3) | all-cis-7,10,13,16,19- |
| (DPA), Clupanodonic acid | | docosapentaenoic acid |
| Docosahexaenoic acid (DHA) | 22:6 (n-3) | all-cis-4,7,10,13,16,19- |
| | | docosahexaenoic acid |
| Tetracosapentaenoic acid | 24:5 (n-3) | all-cis-9,12,15,18,21- |
| | | docosahexaenoic acid |
| Tetracosahexaenoic acid | 24:6 (n-3) | all-cis-6,9,12,15,18,21- |
| (Nisinic Acid) | | tetracosenoic acid |

TABLE 2
Sources of botanical extracts of omega-3 fatty acids

| Common Name | Alternative Name | Linnaean Name | % n-3 |
|---|---|---|---|
| Chia | Chia sage | Salvia hispanica | 64 |
| Kiwifruit | Chinese | Actinidia chinensis | 62 |
| | gooseberry | | |
| Perilla | Shiso | Perilla frutescens | 58 |
| Flax | Linseed | Linum | 55 |
| | | usitatissimum | |
| Lingonberry | Cowberry | Vaccinium vitis- | 49 |
| | | idaea | |
| Camelina | Gold-of-pleasure | Camelina sativa | 36 |
| Purslane | Portulaca | Portulaca oleracea | 35 |
| Black Raspberry | — | Rubus occidentalis | 33 |

Botanical extracts of omega-3 fatty acids may be derived from many different sources. For example, dietary fatty acids containing omega-3 fatty acids may also be derived from algae such as Crypthecodinium cohnii and Schizochytrium, which are rich sources of DHA, or brown algae (kelp) for EPA. Omega-3 fatty acids, or dietary fatty acids, can also be derived from cranberry oil. Dietary fatty acids may also include conjugated linoleic acid (CLA), omega-6 fatty acids, and omega-9 fatty acids, such as linolenic acid, linoleic acid (18:2), and gamma linolenic acid (GLA, 18:3). Vegetarian polyunsaturated omega 3 fatty acids pre-cursors such as stearidonic acid may also be included. Stearidonic acid is a pre-cursor to eicosapentaeonoic acid (EPA) in humans.

A “non-ionic surfactant,” as used herein, is a surface-active agent that tends to be non-ionized (i.e. uncharged) in neutral solutions (e.g., neutral aqueous solutions).

As used herein, the term “oxidation” refers particularly to the degradation or spoiling of an oil or fat through exposure to air or oxygen, resulting in a loss of electrons or an increase in oxidation state. Oxidation can be the result of different chemical mechanisms during the processing, storage, or heating of an oil or fat. There are various types of oxidation, including autooxidation, photosensitized oxidation, thermal oxidation, and enzymatic oxidation. The type of oxidation particularly relevant in this context is thermal oxidation because the formulations and process involved in this application involve heating, and thermal oxidation is one of the most rapid forms of oxidation. Various types of oxidation products are produced by auto-oxidation and thermal oxidation, such as hydroperoxides, aldehydes, and ketones. These degradation products can be measured, providing an analytical index for aging or stability studies for various oils under different conditions, and providing a comprehensive spectrum of decomposition products. The oxidative stability of a fatty acid or lipid may also be determined by methods that are described in the literature (see for example K. Tian and P. Dasgupta, Anal. Chem. 71, 1692-98; 1999, and Firestone, Oxidative Stability Index (OSI): Official Methods of Recommended Practices of the American Oil Chemists Society, 4^{th} Ed. American Oil Chemists Society, Champaign, Ill. Cd 126-92). This method for determining oxidative stability of fats or oils employs the “oxidative stability index” or OSI, which determines the oxidative stability of an oil by passing air through a sample under stringent temperature control. In this technique, a stream of air is passed through the oil sample, which aids in the rapid degradation of the triglyceride into volatile organic acids. The air stream flushes the volatile acids from the oil into a conductivity cell containing water where the acids are solubilized. These acids, once dissolved in the water solution, disassociate into ions, thus changing the conductivity of the water. A constant measure of the conductivity of the cell by computer will indicate when a rapid rise in the conductivity occurs that corresponds to the induction point, which is the oxidative failure of the sample. The OSI time is the time to the induction point. The OSI method has good reproducibility between samples and from laboratory to laboratory. Standards are commercially available, such as saturated fatty acid methyl ester (FAME) from Alltech Associates (Deerfield, Ill.), and can be used to calibrate the OSI determinations. OSI measurements may be performed using an instrument designed to measure oxidative stability manufactured by Omnion (Rockland, Mass.) using the AOCS method described above in the Firestone reference. Fatty acid or oil samples can be run at 110° C. and FAMEs may be tested at 90° C., with air flow set at 35 kPa with resulting velocity of about 140 ml/min. One preferred method for determining OSI values is also described in T. A. Isbell et al., Industrial Crops and Products 9, 115-123 (1999).

As used herein, the term “peroxide value,” or “PV” refers to a quantitative measure of the oxidation of oil. Peroxide value is usually given in meq/Kg of oil (milliequivalents per kilogram). One method used to determine PV is American Oil Chemists' Society Official Method (AOCS) Cd 8-53, as set forth at the date of filing the present disclosure. The peroxide value is also a means of assessing the extent of rancidity reactions that have occurred during storage of a fat or oil. Peroxide value is defined as the amount of peroxide oxygen per kilogram of oil. Peroxide value is measured by determining the amount of iodine which is formed by the reaction of peroxides formed in the oil with iodide ion. A decrease in peroxide values leads to better sensory characteristics or quality of the oil, such as smell and taste.

“Flavonoid” includes compounds that exist in nature and can be divided into the following classes: flavones, flavanones, chalcones, flavon-3-ols, flavan-3-ols, prenylflavonoids, and anthocyanidins. Various flavonoid subclasses include: flavan-3-ols (e.g., proanthocyanidin), flavanones, such as fisetin, flavonols, and flavones, such as apigenin, diosmin, luteolin, nobiletin, tangeretin, anthocyanins, and isoflavones, and other polyphenols (e.g., ellagic acid, resveratrol, and punicalagins). Some representative compounds included: catechins, apigenin, luteolin, naringenin, hesperedin, quercetin, morin, resveratrol, and xanthohumol. The flavonoids are derived from various botanical sources, and can be concentrated by extraction and further purification. In one example, of particular interest is the prenylflavonoid contained in hops, xanthohumol, and the polyphenolic compound, resveratrol, found in grapes and certain other plants.

In further discussion regarding “flavonoids,” these compounds are abundant throughout nature and exert a broad range of biological activities in plants and animals. There are now considered to be over 4,000 flavonoids in nature. Some of the biological activities of flavonoids include: anti-inflammatory, antiviral, antifungal, antibacterial, estrogenic, anti-oxidant, antiallargenic, anticarcinogenic, and antiproliferative medicinal properties.

In one aspect, “hops” (Humulus lupulis L.) has been used for centuries as a bittering agent in the brewing of beer. However, hops is now know to contains alpha acids such as humulone, co-humuone, ad-humulone, and beta acids such as lupulone and co-lupulone. Hops also contains many flavonoids, such as xanthohumol, isoxanthohumol, desmethylxanthohumol, 8-prenylnaringenin, and 6-prenylnaringenin. Xanthohumol is a yellow-orange substance with a melting point of 172° C. and a molecular weight of 354.4. A typical ethanol extract of hops yields about 3 mg/g (3%) of xanthohumol out of a total flavonoid content of 3.46 mg/g. Dried hop contains about 0.2 to 1.0% by weight xanthohumol. Xanthohumol can be extracted and purified to a concentration of 98-99%.

“Resveratrol,” or “trans-resveratrol” (trans-3,4,5-trihydroxystilbene) can be synthesized, or extracted from various plant sources, and is available with a purity of 99%, though other purity levels are available and can be used in accordance with embodiments of the present disclosure.

“Xanthohumol” and other hops prenylflavonoids have been identified as cancer chemopreventive agents through their interfering action with a variety of cellular mechanisms at low micromolar concentrations such as (1) inhibition of metabolic activation of procarcinogens, (2) induction of carcinogen-detoxifying enzymes, and (3) inhibition of tumor growth by inhibiting inflammatory signals and angiogenesis. Ethanol may be used to extract higher levels of the prenylflavonoids from hops. The typical prenylflavonoid content of an ethanol extract of hops includes xanthohumol (3 mg/g), desmethylxanthohumol (0.34 mg/g), isoxanthohumol (0.052 mg/g), 6-prenylnaringenin (0.061 mg/g), and 8-prenylnaringenin 0.015 (mg/g). Supercritical carbon dioxide extractions tend to contain much lower levels, or non-existent levels of prenylflavonoids. In fact, these compounds are almost non-existent in standard CO₂ extracts because the prenylflavonoids are virtually insolvent on carbon dioxide. In the examples provided herein, a xanthohumol extract of purity of 98% has been used, though other extracts can also be used in accordance with embodiments of the present disclosure.

A “prenylflavonoid,” as used herein, refers to a prenylated compound having a substituted or unsubstituted phenol attached to a phenyl via a C₃ alkylene substituted with an oxo group. The C₃ alkylene may be present in a linear chain arrangement (e.g. a chalcone) or joined with other atoms to form a substituted or unsubstituted ring (e.g. a flavanone). Prenylflavonoids may be derived from natural sources (e.g. hops), or synthesized chemically. Tabat et al., Phytochemistry 46: 683-687 (1997).

As used herein, a “prenylated” compound refers to those compounds with an attached —CH₂—CH═C(CH₃)₂ group (e.g. geranylated compounds), optionally hydroxylated prenyl tautomers (e.g. —CH₂—CH—C(CH₃)═CH₂, or —CH₂—C(OH)—C(CH₃)═CH₂), and optionally hydroxylated circularized prenyl derivatives having the formula:

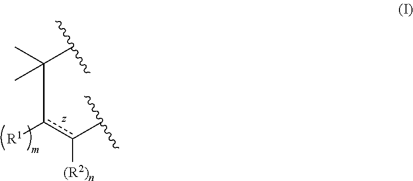

In Formula (I), the dashed bond z represents a double bond or a single bond. R¹ and R² are independently hydrogen or OH. The symbol [symbol] represents the point of attachment to the remainder of the prenylated compounds.

Prenylflavonoids useful in accordance with embodiments of the present disclosure include prenylchalcones and/or prenylflavanones. In some embodiments, the prenylflavonoid is selected from xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, 8-geranylnaringenin, and metabolites and/or derivatives thereof. In some embodiments, the prenylflavonoid can be xanthohumol, a xanthohumol metabolite, or derivative thereof. In some embodiments, the prenylflavonoid is xanthohumol.

The term “transparent water-soluble formulation,” refers to a formulation that can be clearly seen through with the naked eye and is optionally colored. In some embodiments, the transparent water-soluble formulations do not contain particles (e.g. particles of undissolved dietary fatty acid) visible to the naked eye. Thus, in some embodiments, the transparent water-soluble formulations are not opaque, cloudy or milky-white. Transparent water-soluble formulations disclosed herein do not include milky-white emulsions or suspensions in vegetable oil such as corn oil. Transparent water-soluble formulations are also typically not formed by first dissolving the dietary fatty acid in alcohol, or other organic solvents, and then mixed with water.

A “non-alcoholic” formulation, as used herein, is a formulation that does not include (or includes only in trace amounts) methanol, ethanol, propanol or butanol. In other embodiments, the formulation does not include (or includes only in trace amounts) ethanol.

The term “non-aprotic solvated,” as used herein, means that water soluble aprotic solvents are absent or are included only in trace amounts. Water soluble aprotic solvents are water soluble non-surfactant solvents in which the hydrogen atoms are not bonded to an oxygen or nitrogen and therefore cannot donate a hydrogen bond.

“Patient” or “subject” as used herein refers to any mammalian subject, including human subjects.

As used herein, the term “titration” or “trituration” means the slow addition or streaming of a solution to another liquid while mixing. The rate at which the compound or solution is added must not exceed a certain threshold, or the clear nature and viscosity of the solute is lost. Slow addition can be as a drizzle or drop by drop. Slow addition can be specified as a percent of the volume it is being added to per second or per minute, for example 5 ml per second to 100 ml water, or 5% addition per second or minute of the content being added to water or water containing beverage.

As used herein, the term “clear aqueous solution” in reference to a solution containing dietary fatty acid means a water containing solution (e.g. a beverage or other clear solution) that is free of visible particles of undissolved dietary fatty acid or micelles. In some embodiments, the clear aqueous solution is not a visible dispersion, and not a visible suspension, and remains clear upon sitting undisturbed for 1 hour or more. For example, a water-soluble fatty acid formulation according to embodiments disclosed herein may be added to water to form a clear aqueous solution.

The amount of dietary fatty acid adequate to treat a disease or condition is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration. The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient and disease or condition treated. Single or multiple administrations of dietary fatty acid formulations described herein can be administered depending on the dosage and frequency as required and tolerated by a given subject or patient. The formulations should provide a sufficient quantity of active agent to effectively treat the disease state. Lower dosages can be used, particularly when the drug is administered to an anatomically secluded site in contrast to administration orally, into the blood stream, into a body cavity or into a lumen of an organ. Substantially higher dosages can be used in topical administration. Actual methods for preparing parenterally administrable dietary fatty acid formulations will be known or apparent to those skilled in the art and are described in more detail in such publications as Remington's, supra. See also Nieman, In “Receptor Mediated Antisteroid Action,” Agarwal, et al., eds., De Gruyter, New York (1987).

Concentrations, amounts, solubility's, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a concentration range of 0.5 to 400 should be interpreted to include not only the explicitly recited concentration limits of 0.5 and 400, but also to include individual concentrations within that range, such as 0.5, 0.7, 1.0, 5.2, 8.4, 11.6, 14.2, 100, 200, 300, and sub-ranges such as 0.5-2.5, 4.8-7.2, 6-14.9, 55, 85, 100-200, 117, 175, 200-300, 225, 250, and 300-400, etc. This interpretation should apply regardless of the breadth of the range or the characteristic being described.

### II. Water Soluble Formulations

Benefits may be realized from adding nutritional fatty acids such as omega-3 include eicosapentaenoic acid (EPA), docosahexaenoic acid (DHA), fish oil, or flax oil to beverages. Until recently, deodorized fish oils with virtually no fishy taste of smell have not been available. With the availability of deodorized fish oils it is now possible to make beverages containing omega-3 fatty acids, or fish oil, but the stability of the oils remains a serious problem. Normally, these oils are kept frozen to prevent or slow down oxidation. As soon as these oils are defrosted and processed, they begin to undergo oxidation. Oxidation is a natural process that occurs when oils are exposed to air or oxygen. The oxidation of oils can be measured quantitatively by measuring certain markers of oxidation such as the peroxide value (PV), the oxidative stability index (OSI), or isoprostanes, a marker of peroxidation. Rancidification is the oxidation of fats, fatty acids, or edible oils, and most people are familiar with the term rancid to describe the change in smell associated with edible oils or fats such as butter after exposure to air for prolonged periods. A rancid oil or fat also has an objectionable taste. Oxidation is the loss of electrons or increase in oxidation state by a molecule, atom, or ion.

Once oxidized, the undesirable sensory characteristics become apparent. Odor and taste are directly correlated with oxidation. For example, the fishy odor and taste of fish oil is a highly undesirable property of a fish oil-containing beverage. It would be desirable to have a formulation of nutritional fatty acids that were soluble in water containing beverages, or a water-soluble omega-3 fish oil fatty acid formulation that would be stabilized, and virtually free of undesirable odor and taste. In addition, it would also be advantageous to have a process or method for imparting oxidative stability to an oil in need of enhanced oxidative stability, comprising the steps of forming a water-soluble micro-micelle composition consisting of a surfactant, a nutritional fatty acid, a flavonoid or polyphenol, and water, sufficient to impart enhanced stability to the nutritional fatty acid.

It has been discovered that non-ionic surfactants may be used to increase the solubility and/or bioavailability of dietary fatty acids. Thus, non-ionic surfactants may be used to form water-soluble formulations containing dietary fatty acids. It has been further discovered that the addition of a flavonoid such as xanthohumol or a polyphenol such as resveratrol can provide excellent stability and resistance to oxidation to the mixture. This stability is improved over the simple addition of the flavonoid to the fatty acid or lipid (oil).

In one aspect, the present disclosure relates to unique pharmaceutical compositions or water-soluble formulations of a dietary fatty acid, a non-ionic surfactant, a flavonoid or polyphenol, and water. In an alternative embodiment, the water-soluble formulation can be in the form of a stable, water soluble pharmaceutical gel comprising a dietary fatty acid, a non-ionic surfactant, and a flavonoid or polyphenol. In some embodiments, the water-soluble formulation does not include a vegetable oil suspension or visible macro-micelles (micelles visible to the naked eye) in water. In other embodiments, the water-soluble formulation does not include an alcohol (e.g. the dietary fatty acid is not first dissolved in alcohol and then added to water).

The non-ionic surfactant can be a surface-active agent that tends to be non-ionized (i.e. uncharged) in neutral solutions (e.g. neutral aqueous solutions). Useful non-ionic surfactants include, for example, non-ionic water soluble mono-, di-, and tri-glycerides; non-ionic water soluble mono- and di-fatty acid esters of polyethyelene glycol; non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethyleneoxide) triblock copolymers such as POLOXAMER 406 (PLURONIC F-127), and derivatives thereof.

Examples of non-ionic water soluble mono-, di-, and tri-glycerides include propylene glycol dicarpylate/dicaprate (e.g. MIGLYOL 840), medium chain mono- and diglycerides (e.g. CAPMUL and IMWITOR 72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), polyethylene glycol 660 hydroxystearate and derivatives thereof.

Non-ionic water soluble mono- and di-fatty acid esters of polyethyelene glycol include d-α-tocopheryl polyethyleneglycol 1000 succinate (TPGS), poyethyleneglycol 660 12-hydroxystearate (SOLUTOL HS 15), polyoxyl oleate and stearate (e.g. PEG 400 monostearate and PEG 1750 monostearate), and derivatives thereof.

Polyglycolyzed glycerides include polyoxyethylated oleic glycerides, polyoxyethylated linoleic glycerides, polyoxyethylated caprylic/capric glycerides, and derivatives thereof. Specific examples include LABRAFIL M-1944CS, LABRAFIL M-2125CS, LABRASOL, SOFTIGEN, and GELUCIRE.

In some embodiments, the non-ionic surfactant is a glycerol-polyethylene glycol oxystearate, or derivative thereof. These compounds may be synthesized by reacting either castor oil or hydrogenated castor oil with varying amounts of ethylene oxide. Macrogolglycerol ricinoleate is a mixture of 83% relatively hydrophobic and 17% relatively hydrophilic components. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate (glycerol-polyethylene glycol oxysterate) is a mixture of approximately 75% relatively hydrophobic of which a major portion is glycerol polyethylene glycol 12-oxystearate.

In some embodiments, the water-soluble formulations include the dietary fatty acid, a flavonoid or polyphenol, and glycerol-polyethylene glycol oxystearate, to form a transparent water-soluble formulation when admixed in water. The transparent water-soluble formulation can be a formulation that can be clearly seen through with the naked eye and is optionally colored. In some embodiments, the transparent water-soluble formulations do not contain particles (e.g. particles of undissolved dietary fatty acid) visible to the naked eye. Thus, in some embodiments, the transparent water-soluble formulations are not opaque, cloudy or milky-white. Transparent water-soluble formulations disclosed herein do not include milky-white emulsions or suspensions in vegetable oil such as corn oil. Transparent water-soluble formulations are also typically not formed by first dissolving the dietary fatty acid in alcohol, or other organic solvents, and then mixed with water.

In some embodiments, the water-soluble formulation is a non-alcoholic formulation, in that it is a formulation that does not include (or includes only in trace amounts) methanol, ethanol, propanol or butanol. In some embodiments, the formulation does not include (or includes only in trace amounts) ethanol.

The formulation can also be a non-aprotic solvated formulation, in that water soluble aprotic solvents are absent or are included only in trace amounts. Water soluble aprotic solvents are water soluble non-surfactant solvents in which the hydrogen atoms are not bonded to an oxygen or nitrogen and therefore cannot donate a hydrogen bond.

In some embodiments, the water-soluble formulation does not include (or includes only in trace amounts) a polar aprotic solvent. Polar aprotic solvents are aprotic solvents whose molecules exhibit a molecular dipole moment but whose hydrogen atoms are not bonded to an oxygen or nitrogen atom. Examples of polar aprotic solvents include aldehydes, ketones, dimethyl sulfoxide (DMSO), and dimethyl formamide (DMF). In other embodiments, the water soluble formulation does not include (or includes only in trace amounts) dimethyl sulfoxide. Thus, in some embodiments, the water soluble formulation does not include DMSO. In a related embodiment, the water soluble formulation does not include DMSO or ethanol.

In still other embodiments, the water-soluble formulation does not include (or includes only in trace amounts) a non-polar aprotic solvent. Non-polar aprotic solvents are aprotic solvents whose molecules exhibit a molecular dipole of approximately zero. Examples include hydrocarbons, such as alkanes, alkenes, and alkynes.

In some embodiments, the water-soluble formulation consists essentially of dietary fatty acid, a fat-soluble flavonoid, and a non-ionic surfactant. That is, the formulation does not include any water, but optionally may include additional components widely known in the art to be useful in neutraceutical formulations, such as preservatives, taste enhancers, colors, buffers, water, etc. In these formulations, a fat-soluble flavonoid can be dissolved in the surfactant/fatty acid or oil mixture.

In some embodiments, the water-soluble formulation is a water-solubilized formulation, i.e. it includes a dietary fatty acid, a fat soluble flavonoid, a non-ionic surfactant, and water (e.g. a water-containing liquid) but does not include organic solvents (e.g. ethanol). The surfactant/fatty acid/fat soluble flavonoid/water complex can self-assemble into micelles, once a critical concentration is reached. These micelles are invisible to the naked eye, so that, in some embodiments, the water-solubilized formulation is a transparent water-soluble formulation.

Regarding concentrations that can be present in the formulations of the present disclosure, the dietary fatty acid can be present at a concentration of at least about 0.01 mg/ml, at least about 1 mg/ml, at least about 0.01% by weight, or at least about 25% by weight in an alternative embodiment. The total content per dose can be from about 1 mg to about 250 mg, or at least about 10 mg of dietary fatty acid in another embodiment. These concentrations and total content values are based on the formulations that have been solubilized in water, with higher corresponding concentrations being present in gel compositions prior to admixing or titrating with water (depending on how much water is desired or needed to solubilize the gel compositions of the present disclosure). Other concentrations can also be used, as described in further detail below as it relates to dosages and dosage forms.

### III. Methods

In another aspect of the present disclosure, a method of producing stable, water-soluble fatty acid formulations with improved shelf life are provided. Simply warming and mixing the dietary fatty acids with another oil, even if they both have reasonable initial peroxide values, and good sensory characteristics, such as virtually no smell or taste, does not mean it will remain so. This is especially important for fish oils and other similar oils where oxidation will result in a fishy smell and taste, and high PV values.

In addition, if proper procedures are not followed, sometimes a semi-solid gel-like, cloudy or milky, high viscosity solution is obtained, which is often undesirable. This waxy, cloudy, high viscosity gel is often not suitable for forming clear solutions in water or beverages. Rather, it becomes a solidified milky white mass. In contrast, it has been discovered and described herein that by slowly titrating or adding the dietary fatty acid and warm non-ionic surfactant to warm water, a clear solution can be obtained. The rate at which the dietary fatty acid/non-ionic surfactant is added to the warm water and the temperature of each can be central to this process.

In one example, the non-ionic surfactant and water are brought to a certain temperature range of 80-120° F. If the resulting dietary fatty acid/surfactant gel mixture is then added to the water too fast, a solid gel-like mass can result. In a particular embodiment, a dietary fatty acid gel is added to water at a rate of from about 0.05 ml/sec to about 25.0 ml/sec. In another particular embodiment, the temperature of the non-ionic surfactant does not exceed 200° F., and more often is maintained at a temperature of 90 to 120° F. The non-ionic surfactant can be stirred thoroughly to remove bubbles (oxygen), and until clear. In a particular embodiment, once the dietary fatty acid has been added to the non-ionic surfactant, it is stirred for at least 10 minutes, or more, and preferably for about 1 hour. In a more particular embodiment, the water to which it is to be added is heated to about 100 to 150° F. as well, and maintained at about 100° F. while slowly adding the dietary fatty acid gel mixture, though these more specific temperature values are not required.

In one aspect, the present disclosure provides for a more stable formulation of a liquid concentrate or beverage comprising dietary fatty acids, with a low peroxide value, better shelf life characteristics, and enhanced consumer acceptance. For example, a beverage made from fish oil omega-3 fatty acids without a fishy odor or taste, or objectionable sensory qualities. In addition, stable formulations of dietary fatty acids and oils in liquid concentrates or beverages that do not need to be kept frozen to prevent oxidation.

With these general principles in mind, the methods of the present disclosure can provide stable gel compositions of dietary fatty acids with are highly water soluble when added carefully to warm water, or provide clear solutions of dietary fatty acids. In one aspect, a method of stabilizing dietary fatty acids in water can comprise steps of warming a non-ionic surfactant and adding a flavonoid or a polyphenol to the non-ionic surfactant and mixing until dissolved. Additional steps include combining a dietary fatty acid with the non-ionic surfactant and flavonoid or polyphenol to form a surfactant-dietary fatty acid-flavonoid or polyphenol mixture, as well as combining the surfactant-dietary fatty acid-flavonoid or polyphenol mixture with water to form stabilized, clear, water-soluble, self-assembled fatty acid solution. It is noted that the surfactant-dietary fatty acid-flavonoid or polyphenol mixture with water has better shelf life and reduced oxidation during processing and storage (aging), and can be used as a liquid concentrate to be sub-sequentially added to additional water or other liquid.

In another embodiment, a method of making a stable, water-soluble pharmaceutical gel composition of dietary fatty acids can comprise steps of heating a water-soluble non-ionic surfactant in a container to a temperature of about 90° F. to about 200° F. while mixing the non-ionic surfactant until a clear non-ionic surfactant is formed; and adding a flavonoid or polyphenol to the clear non-ionic surfactant and mixing until a clear non-ionic surfactant-flavonoid or polyphenol combination is formed. An additional step can include adding a dietary fatty acid to the clear non-ionic surfactant-flavonoid or polyphenol combination and stirring until thoroughly mixed so as to constitute from 0.1 wt % to 25 wt % dietary fatty acid, from 70 wt % to 99.9 wt % surfactant, and from 0.01 wt % to 5 wt % flavonoid or polyphenol, wherein the dietary fatty acid and flavonoid or polyphenol is sufficiently dispersed or dissolved in the surfactant so that a gel composition is formed containing no visible micelles or particles of dietary fatty acid.

In further detail as it relates to certain steps above for the various embodiments, it is noted that the non-ionic surfactant-dietary fatty acid mixture is typically added at a rate not to exceed 5 ml per second to a volume of water of 100 ml., or not more than 5% of the volume of water per second of the volume of water it is being added to. The rate of addition can also depend to some degree on the volume of water. The water is to be stirred continuously while the addition of the dietary fatty acid gel is being slowly added. The solution may be heated to increase solubility. The heating temperature is typically selected to avoid chemical breakdown of the dietary fatty acid and/or non-ionic surfactant. The temperature of the dietary fatty acid gel (dietary fatty acid/non-ionic surfactant) will typically not exceed 200° F., and the water temperature often will also not exceed 200° F. In one specific embodiment, the temperature of both should be maintained at between 90 and 120° F. In some embodiments, the resulting solution is a water-soluble formulation or transparent water soluble formulation as described above. For example, the resulting solution may be a water soluble formulation that is a crystal clear solution, with no particles visible to the naked eye. Alternatively, the gel composition (prior to addition with water) will be combinable with warm water, as described above, to form a water soluble formulation.

Also provided is a method of enhancing the stability of a dietary fatty acid in a beverage, comprising the steps of combining a dietary fatty acid, a non-ionic surfactant with a flavonoid or polyphenol, and water to form a surfactant-dietary fatty acid-flavonoid-water mixture. Again, the dietary fatty acid can be an omega-3 fatty acid, such as EPA and DHA, and the other ingredients can be present as described herein.

A method of delivering a dietary fatty acid to a subject can also comprise administering a composition as described herein in the form of a beverage to the subject. The composition or formulation typically includes a dietary fatty acid, a non-ionic surfactant; a flavonoid or polyphenol; and water, as described at length herein.

### IV. Dosages and Dosage Forms

According to embodiments disclosed herein, the water-soluble formulations typically include, at a minimum concentration, of about 0.01% by weight, e.g., from about 0.01% to about 35% by weight dietary fatty acid. In another embodiment, the dietary fatty acid can be present in the water-soluble formulation at a concentration from 1 wt % to 35 wt %. In more specific embodiments, the dietary fatty acid can be present in the water-soluble formulation at a concentration from 5 wt % to 30 wt %, or more specifically from 10 wt % to 25 wt %, or still more specifically from 20 wt % to 25 wt %. In one specific embodiment, the dietary fatty acid can be present at a minimum concentration of 25% by weight. These concentrations relate primarily to the formulations that have already been admixed with water, with correspondingly higher concentrations being present in gel composition formulations.

With specific reference to water-solubilized formulations that are ready for drinking in a beverage, the dietary fatty acid may be present (e.g. in a beverage formulation) at a concentration from 0.5 to 1,000 mg per 8 fluid oz. beverage, or alternatively around 1 to 100 mg per ml in a liquid concentrate. In other embodiments, the dietary fatty acid can be present at a concentration from 0.01 mg/ml to 70 mg/ml. In an aspect of the embodiments herein, there can be a maximum concentration for achieving a crystal clear solution. Concentrations of dietary fatty acid above 0.70% (70 mg/ml) using glycerol-polyethylene glycol oxystearate (i.e. macrogoglycerol hydroxystearate) for example, as the surfactant, will no longer result in a crystal-clear solution in water. Therefore, for dietary fatty acids, the concentration range would be from 0.1% to 25% by weight in the surfactant, or 0.01 mg/ml to 250 mg/ml, with one exemplary concentration around 50 mg/ml. This represents a ratio of dietary fatty acid to surfactant of about 1:4, though ranges from 1:1 to 1:10 by weight provides an exemplary range that is useful in some embodiments. In some concentrated formulations (e.g. a soft gel capsule formulation), dietary fatty acid may be present at about 1 to 50 mg/ml, or around 20 mg/ml, or at least 1 mg/ml.

In other embodiments, dietary fatty acid is present in the water-soluble beverage formulation in a minimum amount of from about 0.1 mg to about 1 g. In another embodiment, the dietary fatty acid is present in the water-soluble formulation in an amount from 0.1 mg to 2 g. In a more specific embodiment, from 0.5 mg to 1 g, or more specifically from 1 mg to 500 mg, or still more specifically from 1 mg to 50 mg, or still more specifically from 1 mg to 5 mg of dietary fatty acid can be present in the water-soluble beverage formulation. The flavonoid or polyphenol can be present in an amount of from 1 mg to 500 mg in a solution of 50 ml to 500 ml of non-ionic surfactant. For example, 250 mg of xanthohumol can be dissolved in 50 ml surfactant, and 12 ml of dietary fatty acid can be added to this mixture totaling 62 ml. This is then added to 100 ml of warm water. The total volume of the water-soluble concentrate is then about 162 ml, so the level of the flavonoid would be about 1.54 mg/ml or 0.15%. The flavonoid may be present at a level of from 0.01 wt % to 5 wt %. Likewise, if the flavonoid can be a prenylflavonoid, such as xanthohumol or an analogue of xanthohumol, and the concentration may be from 0.001 wt % to 1% wt %, or alternatively from 0.01 wt % to 1 wt %, or still alternatively from 0.01% to 0.5% in solution of water-soluble concentrate.

These concentrations, as well as all others described herein, are merely exemplary, as any concentrations can be used provided they are capable of providing clear, stable solutions when admixed with water. Thus, there are multiple formulations disclosed that are useful in accordance with embodiments described herein. For example, a formulation can be solublized in water in a dosage form for drinking or other similar administration. The formulation can include some water, but in more of a concentrated form, such as may be useful for delivery in soft gel capsules or other administration formulations. Still further, the formulation can include a gel formulation, prior to admixture with any appreciable amount of water, which can be administered as a gel or packaged for use by an end user to mix with water.

In some embodiments, the water-soluble formulation can be in the form of a pharmaceutical composition. The pharmaceutical composition may include dietary fatty acid such as fish oil omega-3 fatty acids, a non-ionic surfactant, a prenylflavonoid such as xanthohumol, and a pharmaceutically acceptable excipient. After a pharmaceutical composition, including dietary fatty acid, has been formulated in an acceptable carrier, it can be placed in an appropriate container and labeled for treatment of an indicated condition. For administration of dietary fatty acid, such labeling may include, for example, instructions concerning the amount, frequency and method of administration.

Any appropriate dosage form is useful for administration of the water-soluble formulation of the present invention, such as oral, parenteral and topical dosage forms. Oral preparations include tablets, pills, powder, dragees, capsules (e.g. soft-gel capsules), liquids, lozenges, gels, syrups, slurries, beverages, suspensions, etc., suitable for ingestion by the patient. Examples of liquid formulations are drops, sprays, aerosols, emulsions, lotions, suspensions, drinking solutions, gargles, and inhalants. The formulations of the present disclosure can also be administered by injection, that is, intravenously, intramuscularly, intracutaneously, subcutaneously, intraduodenally, or intraperitoneally. Also, the formulations described herein can be administered by inhalation, for example, intranasally. Additionally, the formulations of the present disclosure can be administered transdermally. The formulations can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Thus, the formulations described herein may be adapted for oral administration.

For preparing pharmaceutical compositions from the formulations of the present disclosure, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

Suitable carriers include magnesium carbonate, magnesium stearate, talc, sugar, lactose, pectin, dextrin, starch (from corn, wheat, rice, potato, or other plants), gelatin, tragacanth, a low melting wax, cocoa butter, sucrose, mannitol, sorbitol, cellulose (such as methyl cellulose, hydroxypropylmethyl-cellulose, or sodium carboxymethylcellulose), and gums (including arabic and tragacanth), as well as proteins such as gelatin and collagen. If desired, disintegrating or co-solubilizing agents may be added, such as the cross-linked polyvinyl pyrrolidone, agar, alginic acid, or a salt thereof, such as sodium alginate. In powders, the carrier is a finely divided solid, which is in a mixture with the finely divided active component. In tablets, the active component is mixed with the carrier having the necessary binding properties in suitable proportions and compacted in the shape and size desired.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain dietary fatty acid mixed with a filler or binders such as lactose or starches, lubricants such as talc or magnesium stearate, and, optionally, stabilizers. In soft capsules, dietary fatty acid may be dissolved or suspended in suitable liquids, such as fatty oils, lecithin, phospholipids such as phosphatidylcholine, medium chain triglycerides, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

For parenteral injection, liquid preparations can be formulated in solution in aqueous polyethylene glycol solution.

Aqueous solutions and beverages suitable for oral use can be prepared by dissolving the active component in water and adding suitable colorants, flavors, stabilizers, and thickening agents as desired. Aqueous suspensions suitable for oral use can be made by dispersing the active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. These preparations may contain, in addition to the active component, colorants, flavors, stabilizers, buffers, artificial and natural sweeteners, dispersants, thickeners, solubilizing agents, and the like.

Sweetening agents can be added to provide a palatable oral preparation, such as glycerol, sorbitol or sucrose. As an example of an injectable oil vehicle, see Minto, J. Pharmacol. Exp. Ther. 281:93-102, 1997. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The formulations of the disclosure can be delivered transdermally, by a topical route, formulated as applicator sticks, solutions, suspensions, emulsions, gels, creams, ointments, pastes, jellies, paints, powders, and aerosols.

The formulations can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the formulations of the present disclosure can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the dietary fatty acid, dietary fatty acid metabolite, flavonoid, xanthohumol, or salt thereof into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The formulations may be administered as a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

### Assays

Subject non-ionic surfactants may be assayed for their ability to solubilize dietary fatty acid using any appropriate method. Typically, a non-ionic surfactant is warmed and contacted with the dietary fatty acid and mixed mechanically and/or automatically using a shaker, vortex, or sonicator device. Water may be optionally added, for example, where the dietary fatty acid and/surfactant/flavonoid is to be incorporated into a beverage. The solution is heated to increase solubility. The heating temperature is selected to avoid chemical breakdown of the dietary fatty acid or non-ionic surfactant. In a particular example, the surfactant or dietary fatty acid is not heated above 200 degrees F., and preferably not more than 150 degrees F. Ideally, the temperature is maintained at about 90-100° F.

The resulting solution may be visually inspected for colloidal particles to determine the degree of solubility of the dietary fatty acid. Alternatively, the solution may be filtered and analyzed to determine the degree of solubility. For example, a spectrophotometer may be used to determine the concentration of dietary fatty acid present in the filtered solution. Typically, the test solution is compared to a positive control containing a series of known quantities of pre-filtered dietary fatty acid solutions to obtain a standard concentration versus UV/vis absorbance curve. Alternatively, high performance liquid chromatography may be used to determine the amount of dietary fatty acid in solution. Micelles in a size range of from 10 to 100 nm can be measured by light scattering experiments. Typical sizes are from 10 to 50 nm for fatty acid self assembled micelles formed by this invention.

Oxidative stability assay methods are well known in the art. Typically, these methods involve automated dispensing and mixing of solutions with varying amounts of non-ionic surfactants, dietary fatty acid, flavonoid, and water, and optionally other co-solvents. The resulting solutions may then be analyzed to determine the degree of oxidative stability using any appropriate method as discussed above.

For example, as mentioned previously, the oxidative stability of a fatty acid may be determined by methods that are described in the literature (see for example K. Tian and P. Dasgupta, Anal. Chem. 71, 1692-98; 1999, and Firestone, Oxidative Stability Index (OSI): Official Methods of Recommended Practices of the American Oil Chemists Society, 4^{th} Ed. American Oil Chemists Society, Champaign, Ill. Cd 126-92). This method for determining oxidative stability of fats or oils employs the “oxidative stability index” or OSI, which determines the oxidative stability of an oil by passing air through a sample under stringent temperature control. In this technique, a stream of air is passed through the oil sample, which aids in the rapid degradation of the triglyceride into volatile organic acids. The air stream flushes the volatile acids from the oil into a conductivity cell containing water where the acids are solubilized. These acids, once dissolved in the water solution, disassociate into ions, thus changing the conductivity of the water. A constant measure of the conductivity of the cell by computer will indicate when a rapid rise in the conductivity occurs that corresponds to the induction point, which is the oxidative failure of the sample. The OSI time is the time to the induction point. The OSI method has good reproducibility between samples and from laboratory to laboratory. Standards are commercially available, such as saturated fatty acid methyl ester (FAME) from Alltech Associates (Deerfield, Ill.), and can be used to calibrate the OSI determinations. OSI measurements may be performed using an instrument designed to measure oxidative stability manufactured by Omnion (Rockland, Mass.) using the AOCS method described above in the Firestone reference. Fatty acid or oil samples can be run at 110° C. and FAMEs may be tested at 90° C., with air flow set at 35 kPa with resulting velocity of about 140 ml/min. One preferred method for determining OSI values is also described in T. A. Isbell et al., Industrial Crops and Products 9, 115-123 (1999).

Alternatively, one can measure oxidative stability by measuring the peroxide value (PV) according to the methods mentioned previously.

Thus, one skilled in the art may test a wide variety of surfactants, flavonoids or polyphenols to determine their ability to provide oxidative stability to dietary fatty acid compounds.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the formulations are equally applicable to the methods of treating disease states described herein. All publications, patents, and patent applications cited herein are hereby incorporated by reference in their entirety for all purposes.

### EXAMPLES

The examples below are meant to illustrate certain embodiments of the present disclosure, and are not intended to limit the scope of the invention.

### Example 1

Water-soluble compositions of omega-3 fatty acids are formulated containing the non-ionic surfactant macrogolglycerol hydroxystearate (Glycerol-Polyethylene glycol oxystearate). First, the non-ionic surfactant was heated to about 100° F. and stirred until clear and virtually no bubbles are apparent. Xanthohumol (98% purity) is mixed into the surfactant until a clear, transparent, yellow gel is formed. A deodorized omega-3 fatty acid fish oil, containing 30% total omega-3 fatty acids at room temperature is very slowly added into the warm macrogolglycerol hydroxystearate until a clear slightly viscous solution is formed containing dissolved omega-3 fatty acids and xanthohumol (hereinafter referred to as “omega-3 gel formulation”). The omega-3 gel formulation consists of macrogolglycerol hydroxystearate (100 ml), 250 mg of xanthohumol (98% purity), 25 ml (25 grams) of omega-3 fatty acids, representing a concentration of 20% or 20 mg/ml for the omega-3 fatty acids in the non-ionic surfactant.

In another vessel, 250 ml of warm water (90° to 100° F.) is maintained, and the non-ionic surfactant, omega-3 fatty acids, and xanthohumol mixture is slowly added to the warm water until dissolved with continuous mixing. The non-ionic surfactant, omega-3 fatty acids, and xanthohumol mixture is slowly titrated at a rate of about 1 ml per second to the 250 ml of warm water that is maintained as a mixing vortex with a stirrer at 100 RPM, and maintained at a temperature of about 100° F. until a crystal clear solution is formed. The water is continuously stirred during the addition phase and after, until a clear liquid is formed. This solution contains self-assembled micelles containing omega-3-fatty-acids, surfactant, xanthohumol, and water.

A solution prepared in accordance with these steps was tested for peroxide value (PV) according the previously described protocol, and found to have a PV value of less than 0.1 meq/Kg. A sample of the same omega-3 fatty acids used in this example that was kept refrigerated after defrosting for 1 week had a PV value of 0.2 meq/Kg right after defrosting, and a PV value of 2.5 meq/Kg within 2 weeks. Another sample kept at room temperature, in a sealed, amber glass container, had a PV value of 4.5 meq/Kg after 30 days.

### Formulation Prepared in Accordance the Procedures Described in the Present Example

| Ingredients | Amount |
|---|---|
| Macrogolglycerol Hydroxystearate | 100 ml |
| Xanthohumol 98% | 250 mg |
| Omega-3 fish oil | 25 ml |
| Water | 250 ml |

As can be seen from the above example, a stable, aqueous solution of solubilized omega-3 fatty acids was achieved by adding the omega-3 fatty acid/xanthohumol gel formulation to the warm water to make a stabilized water soluble beverage. More specifically, the aqueous omega-3 fatty acid/flavonoid formulation was prepared by maintaining the gel formulation at a temperature of about 100° F. and titrating or adding drop by drop the gel mixture to warm water to form a clear aqueous solution of stabilized omega-3 fatty acids. This aqueous omega-3 fatty acid formulation did not have undesirable flavor. The aqueous omega-3 fatty acid formulation consisted of water (250 ml), macrogolglycerol hydroxystearate 40 (100 ml), and 30% omega-3 fatty acid fish oil (25 grams), a concentration of omega-3 fatty acids in the aqueous dietary fatty acid formulation of 6.6% or 66 mg/ml (water containing beverage). The aqueous omega-3 fatty acid formulation was analyzed by HPLC to verify content of total fatty acids. The peroxide value was measured by methods according to Official Methods of Analysis, 15^{th} ed., Association of Official Analytical Chemists: 965.33-peroxides titrated in KI with sodium thiosulfate, and found to be <0.1 meq/kg after 60 days. The same omega-3 fatty acids that were not processed according to this process, and stored under similar conditions, were measured for peroxide value at 60 days with a PV value of 2.25 meq/kg. The formulation of present example, prepared as described herein, clearly exhibited an enhanced shelf-life or resistance to oxidation over the omega-3 fatty acid samples that were not processed in accordance with the teachings of the present disclosure.

### Example 2

The following formulation was prepared as described below: 5 grams of DHA (docosahexaenoic acid) oil from algae was dissolved in 50 ml of warm Polyethylene Glycol 660 Hydroxystearate and 500 mg of xanthohumol, by mixing until a clear gel was formed. This gel was slowly added to 250 ml of warm water until dissolved, which involved mixing with a paddle suspended and rotating at 50 RPM by slowly adding as a drizzle, or drop-by-drop, using a titration apparatus. The DHA/surfactant/xanthohumol gel was added very slowly to the mixing water to avoid solidification of the liquid into a solid gel, or cloudy white mass. The DHA oil was added at the rate of 1 ml every 10 seconds or more while stirring continues. A clear solution was formed with no visible particles or micelles. This stabilized, water soluble fatty acid solution was tested and found to have a PV value of 0.4 meq/Kg.

### Example 3

About 100 ml of the non-ionic surfactant macrogolglycerol hydroxystearate (Glycerol-Polyethylene glycol oxystearate) is heated to a temperature of 100° C., and mixed until clear. Next, 5 grams of trans-resveratrol (trans-3,4,5-trihydroxystilbene-99% pure) is mixed into the surfactant until fully dissolved or clear. 25 ml of a deodorized omega-3 fatty acid fish oil, containing 30% total omega-3 fatty acids at room temperature is very slowly added into the warm macrogolglycerol hydroxystearate until a clear slightly viscous solution is formed containing dissolved omega-3 fatty acids and xanthohumol. In another vessel, 200 ml of water is also heated and maintained at 100° C. Additionally, 1 gram of ascorbic acid is added to the water and dissolved. The non-ionic surfactant/resveratrol mixture is slowly added to the warm water and was constantly mixed or stirred until the surfactant/resveratrol/fish oil mixture is fully incorporated into the water.

While the disclosure has been described with reference to certain preferred embodiments, those skilled in the art will appreciate that various modifications, changes, omissions, and substitutions can be made without departing from the spirit of the disclosure. It is therefore intended that the invention be limited only by the scope of the appended claims.

What is claimed is:

1. A stable fatty acid-containing formulation, comprising:
a dietary fatty acid;
a non-ionic surfactant;
a flavonoid or polyphenol; and
water.

2. The formulation of claim 1, wherein the dietary fatty acid is an omega-3 fatty acid.

3. The formulation of claim 1, wherein the omega-3 fatty acid is eicosapentaenoic acid (EPA), docosahexaenoic acid (DHA), or combination thereof.

4. The formulation of claim 1, wherein the formulation is a non-alcoholic formulation.

5. The formulation of claim 1, wherein the formulation is a non-aprotic solvated formulation.

6. The formulation of claim 1, wherein the dietary fatty acid is present at a concentration of at least about 0.01 mg/ml.

7. The formulation of claim 1, wherein the dietary fatty acid is present at a concentration of at least about 1 mg/ml.

8. The formulation of claim 1, wherein the dietary fatty acid is present at a concentration of at least about 0.01% by weight.

9. The formulation of claim 1, wherein the dietary fatty acid is present at a concentration of at least about 25% by weight.

10. The formulation of claim 1, comprising from about 1 mg to about 250 mg of the dietary fatty acid.

11. The formulation of claim 1, comprising at least about 10 mg of the dietary fatty acid.

12. The formulation of claim 1, wherein the non-ionic surfactant is selected from the group consisting of non-ionic water soluble mono-, di-, or triglycerides; non-ionic water soluble mono- or di-fatty acid esters of polyethylene glycol; non-ionic water soluble sorbitan fatty acid esters; polyglycolyzed glycerides; non-ionic water soluble triblock copolymers; and derivatives thereof.

13. The formulation of claim 1, wherein the non-ionic surfactant is a non-ionic water-soluble mono-, di-, or tri-glyceride.

14. The formulation of claim 1, wherein the non-ionic surfactant is a glycerol-polyethylene glycol oxystearate.

15. The formulation of claim 1, wherein the non-ionic surfactant is a macrogolglycerol ricinoleate, a macrogolglycerol hydroxystearate, or a polyethylene glycol 660 hydroxystearate.

16. The formulation of claim 1, wherein the non-ionic surfactant is a polyethylene glycol 660 hydroxystearate.

17. The formulation of claim 1, wherein the flavonoid or polyphenol is a xanthohumol, a resveratrol, or a salt thereof.

18. The formulation of claim 1, wherein the formulation is an oral formulation.

19. The formulation of claim 18, wherein the oral formulation is a soft gel capsule.

20. The formulation of claim 18, wherein the oral formulation is a beverage.

21. The formulation of claim 1, wherein the formulation is a spray formulation.

22. The formulation of claim 1, wherein the formulation is a topical formulation.

23. The formulation of claim 1, wherein the dietary fatty acid is derived from a fish, algae, or vegetable source.

24. The formulation of claim 1, in which a peroxide value does not exceed about 2.0 meq/kg during one week of storage.

25. A method of stabilizing dietary fatty acids in water, comprising:
warming a non-ionic surfactant;
adding a flavonoid or a polyphenol to the non-ionic surfactant and mixing until dissolved;
combining a dietary fatty acid with the non-ionic surfactant and flavonoid or polyphenol to form a surfactant-dietary fatty acid-flavonoid or polyphenol mixture; and
combining the surfactant-dietary fatty acid-flavonoid or polyphenol mixture with water to form stabilized, clear, water-soluble, self-assembled fatty acid solution.

26. The method of claim 25, wherein the temperature is from about 90° F. to about 200° F.

27. The method of claim 25, wherein a rate of addition is from about 0.05 ml/sec to about 25.0 ml/sec for at least one step.

28. The method of claim 25, wherein a rate of addition is from about 0.05 ml/sec to about 25.0 ml/sec for all steps.

29. The method of claim 25, wherein the solution is a concentrate.

30. The method of claim 25, wherein said non-ionic surfactant is a glycerol-polyethylene glycol oxystearate, an ethoxylated castor oil, or a polyethylene glycol 660 hydroxystarate, and the flavonoid or polyphenol compound is xanthohumol or resveratrol.

31. A water soluble formulation, comprising:
a dietary fatty acid;
a non-ionic surfactant; and
a flavonoid or polyphenol,
wherein the formulation is in the form of a stable, water-soluble pharmaceutical gel.

32. The formulation of claim 31, wherein the dietary fatty acid is an omega-3 fatty acid.

33. The formulation of claim 32, wherein the omega-3 fatty acid is eicosapentaenoic acid (EPA), docosahexaenoic acid (DHA), or combination thereof.

34. The formulation of claim 31, wherein the formulation is a non-alcoholic and non-aprotic solvated formulation.

35. The formulation of claim 31, wherein the non-ionic surfactant is selected from the group consisting of non-ionic water soluble mono-, di-, or tri-glycerides; non-ionic water soluble mono- or di-fatty acid esters of polyethylene glycol; non-ionic water soluble sorbitan fatty acid esters; polyglycolyzed glycerides; non-ionic water soluble triblock copolymers; and derivatives thereof.

36. The formulation of claim 31, wherein the non-ionic surfactant is a glycerol-polyethylene glycol oxystearate, a macrogolglycerol ricinoleate, a macrogolglycerol hydroxystearate, or a polyethylene glycol 660 hydroxystearate.

37. The formulation of claim 31, wherein the flavonoid or polyphenol is a xanthohumol, a resveratrol, or a salt thereof.

38. A method of making a stable, water-soluble pharmaceutical gel composition of dietary fatty acids, comprising the steps:
heating a water-soluble non-ionic surfactant in a container to a temperature of about 90° F. to about 200° F. while mixing the non-ionic surfactant until a clear non-ionic surfactant is formed;
adding a flavonoid or polyphenol to the clear non-ionic surfactant and mixing until a clear non-ionic surfactant-flavonoid or polyphenol combination is formed; and
adding a dietary fatty acid to the clear non-ionic surfactant-flavonoid or polyphenol combination and stirring until thoroughly mixed so as to constitute from 0.1 wt % to 25 wt % dietary fatty acid, from 70 wt % to 99.9 wt % surfactant, and from 0.01 wt % to 5 wt % flavonoid or polyphenol, wherein the dietary fatty acid and flavonoid or polyphenol is sufficiently dispersed or dissolved in the surfactant so that a gel composition is formed containing no visible micelles or particles of dietary fatty acid.

39. The method of claim 38, further comprising the step of adding the gel composition to warm water at a rate not to exceed 5% of the volume of water per second while continuously stirring the water until a clear, stabilized solution is formed.

40. The method of claim 38, wherein the non-ionic surfactant is a glycerol-polyethylene glycol oxystearate, an ethoxylated castor oil, or a polyethylene glycol 660 hydroxystarate, and is flavonoid is xanthohumol.

41. The method of claim 38, wherein the polyphenol is a resveratrol or a trans-resveratrol.

42. The method of claim 38, wherein said non-ionic surfactant is a glycerol-polyethylene glycol oxystearate, an ethoxylated castor oil, or a polyethylene glycol 660 hydroxystarate, and said flavonoid is xanthohumol.

43. The method of claim 38, wherein the polyphenol is a resveratrol or a trans-resveratrol.

44. A method of enhancing the stability of a dietary fatty acid in a beverage, the method comprising the steps of combining a dietary fatty acid, a non-ionic surfactant, a flavonoid or polyphenol, and water to form a clear, stable surfactant-dietary fatty acid-flavonoid-water mixture.

45. The method of claim 44, wherein the dietary fatty acid is an omega-3 fatty acid.

46. The method of claim 45, wherein the omega-3 fatty acid is one or more of EPA and DHA.

47. A method of delivering a dietary fatty acid to a subject comprising administering the composition of claim 1 to the subject.

48. The method of claim 47, wherein the composition is administered orally to the subject.

49. The method of claim 47, wherein the composition is administered in the form of a soft capsule to the subject.

50. The method of claim 47, wherein the composition is administered in the form of a beverage to the subject.
